(19) United States
(12) Patent Application Publication
(10) Pub. No.: US 2024/0423895 A1
(43) Pub. Date: Dec. 26, 2024

(54) AEROSOL TYPE CREAM HAIR DYE COMPOSITION

(71) Applicant: DARIYA CO., LTD., Nagoya-shi (JP)

(72) Inventor: Kenji IRIAI, Inazawa-shi (JP)

(21) Appl. No.: 18/827,009

(22) Filed: Sep. 6, 2024

Related U.S. Application Data
(63) Continuation of application No. PCT/JP2023/019365, filed on May 24, 2023.

(51) Int. Cl. A61K 8/86; A61K 8/31; A61K 8/34; A61Q 5/10

(52) U.S. Cl. CPC A61K 8/86; A61K 8/31; A61K 8/342; A61Q 5/10; A61K 2800/5422; A61K 2800/591

(57) ABSTRACT

An aerosol type cream hair dye composition, in which a first agent and a second agent have good simultaneous dispensability and good mixability after long-term storage, is provided. The aerosol type cream hair dye composition includes the first agent containing an alkaline agent and an oxidation dye, and the second agent containing an oxidizing agent, wherein both the first agent and the second agent contain (A) a nonionic surfactant, (B) a higher alcohol having 12 to 24 carbon atoms, and (C) a hydrocarbon that is in a liquid or paste form at 25° C.

### CROSS-REFERENCE TO RELATED APPLICATION

This application is a continuation application of PCT Application No. PCT/JP2023/019365, filed on May 24, 2023, which is incorporated by reference for all purposes.

### BACKGROUND

The present disclosure relates to an aerosol type cream hair dye composition, and particularly relates to an aerosol type cream hair dye composition, in which a first agent and a second agent have good simultaneous dispensability and good mixability after long-term storage.

With the increase in fashion consciousness in recent years, oxidative hair dyes (hereafter referred to as hair dyes) have become widely used as a method to change the appearance of hair. A hair dye exhibits excellent color development and color tone persistence through the use of the mixture of a first agent containing an alkali agent and an oxidative dye and a second agent containing an oxidizing agent, which are mixed upon use. Although there are a wide variety of hair dye formulations, aerosol type hair dyes are known as a formulation whereby the first agent and the second agent may be mixed in small quantities for use in multiple times, and the remainder may be stored until the next hair dye application, and the first agent and the second agent may be easily dispensed at a specified ratio (hereinafter referred to as “simultaneous dispensability”). Furthermore, cream preparations are widely used for aerosol type hair dyes from the viewpoint of usability and staining. Since aerosol type cream hair dyes may be stored for a long period of time for export, etc., these hair dyes are required to have not only good simultaneous dispensability immediately after production, but also good simultaneous dispensability after long-term storage. An example of such aerosol type cream hair dye having good simultaneous dispensability after long-term storage is the one in Japanese Patent Laid-Open No. 2004-91355.

In Japanese Patent Laid-Open No. 2004-91355, simultaneous dispensability after long-term storage has been studied, but the mixability of the first agent and the second agent after long-term storage has not been studied.

Therefore, an objective of the present disclosure is to provide an aerosol type cream hair dye composition, in which a first agent and a second agent have good simultaneous dispensability and good mixability after long-term storage.

### SUMMARY

As a result of intensive research into solving the above problems, the present inventors have discovered that an aerosol type cream hair dye composition including a first agent containing an alkaline agent and an oxidative dye and a second agent containing an oxidizing agent, wherein both the first agent and the second agent contain: (A) a nonionic surfactant, (B) a higher alcohol having 12 to 24 carbon atoms, and (C) a hydrocarbon that is in a liquid or paste form at 25° C.; and have good simultaneous dispensability and good mixability after long-term storage. Hence, the present inventors have completed the present disclosure.

The present disclosure provides an aerosol type cream hair dye composition, in which the first agent and the second agent have good simultaneous dispensability and good mixability after long-term storage.

### DETAILED DESCRIPTION

The present disclosure is described in detail below. All units indicating contents are mass % unless otherwise specified.

The long-term storage in the present disclosure means to store for six months under 40° C. conditions.

The present disclosure relates to an aerosol type cream hair dye composition including a first agent containing an alkaline agent and an oxidative dye and a second agent containing an oxidizing agent, wherein both the first agent and the second agent contain (A) a nonionic surfactant, (B) a higher alcohol having 12 to 24 carbon atoms, and (C) a hydrocarbon that is in a liquid or paste form at 25° C.

The first agent of the aerosol type cream hair dye composition in the present disclosure is described.

The first agent of the present disclosure contains nonionic surfactant (A) from the viewpoint of the simultaneous dispensability of the first agent and the second agent after long-term storage.

Examples of ingredient (A) above used in the first agent of the present disclosure include, but are not particularly limited to, polyoxyethylene (hereinafter referred to as POE) alkyl ether, POE polyoxypropylene (hereinafter referred to as POP) alkyl ether, POE fatty acid ester, POE sorbitan fatty acid ester, POE castor oil, POE hardened castor oil, POE sorbitol tetra fatty acid ester, POE phytosterol, glycerol fatty acid ester, sorbitan fatty acid ester, polyglycerol fatty acid ester, sucrose fatty acid ester, alkyl polyglucoside, N-alkyl dimethyl amine oxide, and alkanolamide, and one or two or more types thereof may be contained.

Among the examples of ingredient (A) above used in the first agent of the present disclosure, POE alkyl ether is preferred from the viewpoint of the simultaneous dispensability of the first agent and the second agent after long-term storage.

Examples of the POE alkyl ether as ingredient (A) above used in the first agent of the present disclosure include, but are not particularly limited to, POE (2) lauryl ether, POE (4.2) lauryl ether, POE (9) lauryl ether, POE (21) lauryl ether, POE (25) lauryl ether, POE (2) cetyl ether, POE (5) cetyl ether, POE (5.5) cetyl ether, POE (7) cetyl ether, POE (8) cetyl ether, POE (10) cetyl ether, POE (13) cetyl ether, POE (15) cetyl ether, POE (20) cetyl ether, POE (23) cetyl ether, POE (25) cetyl ether, POE (30) cetyl ether, POE (40) cetyl ether, POE (2) stearyl ether, POE (4) stearyl ether, POE (11) stearyl ether, POE (15) stearyl ether, POE (20) stearyl ether, POE (30) stearyl ether, POE (40) stearyl ether, POE (2) oleyl ether, POE (7) oleyl ether, POE (10) oleyl ether, POE (15) oleyl ether, POE (20) oleyl ether, POE (50) oleyl ether, POE (5) behenyl ether, POE (10) behenyl ether, POE (20) behenyl ether, and POE (30) behenyl ether, and one or two or more types thereof may be contained. Of these, one or more types selected from POE (20) stearyl ether or POE (5) behenyl ether are preferred from the viewpoint of the simultaneous dispensability of the first agent and the second agent after long-term storage. The numerals in parentheses following POE each refer to the number of moles of ethylene oxide added.

From the viewpoint of the simultaneous dispensability of the first agent and the second agent after long-term storage, two or more types of ingredient (A) above used in the first agent of the disclosure are preferably contained, and more preferably POE (20) stearyl ether and POE (5) behenyl ether are used in combination.

The content of ingredient (A) above used in the first agent of the present disclosure is not particularly limited, but ranges from preferably 1% to 8%, and more preferably 2.1% to 8%. If the content of ingredient (A) above is less than 1%, the simultaneous dispensability of the first agent and the second agent may deteriorate after long-term storage. If the content of ingredient (A) above exceeds 8%, no further effect may be obtained.

The first agent of the present disclosure contains higher alcohol (B) having 12 to 24 carbon atoms from the viewpoint of the simultaneous dispensability and the mixability of the first agent and the second agent after long-term storage.

Examples of ingredient (B) above used in the first agent of the present disclosure include, but are not particularly limited to, lauryl alcohol, myristyl alcohol, cetanol, stearyl alcohol, cetostearyl alcohol, isostearyl alcohol, oleyl alcohol, arachyl alcohol, behenyl alcohol, octyldodecanol, and decyltetradecanol, and one or two more thereof may be contained. Of these, cetanol and cetostearyl alcohol are preferably used in combination from the viewpoint of the simultaneous dispensability of the first agent and the second agent after long-term storage.

The content of ingredient (B) above used in the first agent of the present disclosure is not particularly limited, but ranges from preferably 0.5% to 5.5%, and more preferably 1.3% to 4%. If the content of ingredient (B) above is less than 0.5% or more than 5.5%, the mixability of the first agent and the second agent may deteriorate after long-term storage.

The first agent of the present disclosure contains hydrocarbon (C) that is in a liquid or paste form at 25° C., from the viewpoint of the simultaneous dispensability and the mixability of the first agent and the second agent after long-term storage.

Regarding ingredient (C) above used in the first agent of the present disclosure, examples of the hydrocarbon that is in a liquid form at 25° C. include, but are not particularly limited to, liquid paraffin, liquid isoparaffin, light liquid isoparaffin, heavy liquid isoparaffin, squalane, and vegetable squalane, and examples of the hydrocarbon that is in a paste form at 25° C. include, but are not particularly limited to, vaseline, and one or two or more types thereof may be contained. Of these, liquid paraffin, liquid isoparaffin, and squalane are preferred from the viewpoint of the simultaneous dispensability of the first agent and the second agent after long-term storage, and liquid paraffin is more preferred from the viewpoint of the mixability of the first agent and the second agent after long-term storage.

The content of ingredient (C) above used in the first agent of the present disclosure is not particularly limited, but preferably ranges from 0.1% to 15%, more preferably 0.1% to 10%, and even more preferably 1% to 10%. If the content of ingredient (C) above is less than 0.1%, the mixability of the first agent and the second agent may deteriorate after long-term storage. If the content of ingredient (C) above exceeds 15%, the simultaneous dispensability and the mixability of the first agent and the second agent may deteriorate after long-term storage.

The first agent of the present disclosure contains an alkaline agent from the viewpoint of hair dyeability.

Examples of the alkaline agent used in the first agent of the present disclosure include, but are not particularly limited to, ammonia, monoethanolamine, ammonium hydrogencarbonate, sodium hydrogencarbonate, and sodium carbonate, and one or two or more types thereof may be contained.

The content of the alkaline agent used in the first agent of the present disclosure is not particularly limited, but preferably ranges from 0.1% to 10%.

The first agent of the hair dye composition in the present disclosure may contain an oxidative dye from the viewpoint of hair dyeability.

Examples of the oxidative dye used in the first agent of the present disclosure include, but are not particularly limited to, 2,4-diaminophenoxyethanol, toluene-2,5-diamine, para-aminophenol, paraphenylenediamine, meta-aminophenol, metaphenylenediamine, resorcin, 5-amino-orthocresol and salts thereof, and one or two or more types thereof may be contained. Specific examples of salts of these oxidative dyes include hydrochlorides and sulfates.

The content of the oxidative dye used in the first agent of the present disclosure is not particularly limited, but preferably ranges from 0.01% to 10%.

The viscosity of the first agent of the present disclosure at 20° C. is not particularly limited, but preferably ranges from 100 mPa·s to 100,000 mPa·s.

The viscosity of the first agent of the present disclosure at 20° C. was measured by filling a sample bottle (Food 140: Manufactured by Dai-ichi Glass Co., Ltd.) with 120 g of the first agent prepared by a usual method and allowing it to stand at 20° C. for 24 hours, and then rotating it for 1 minute: at 60 rpm using an M4 rotor when the viscosity was 100 mPa·s or more and less than 10,000 mPa·s as measured with a B-type viscometer (Model: Digital Viscometer TVB-10M, manufactured by Toki Sangyo Co., Ltd); at 30 rpm using the M4 rotor when the viscosity was 10,000 mPa·s or more and less than 20,000 mPa·s as measured with the same; at 12 rpm using the M4 rotor when the viscosity was 20,000 mPa·s or more and less than 50,000 mPa·s as measured with the same; or at 6 rpm using the M4 rotor when the viscosity was 50,000 mPa·s or more and 100,000 mPa·s or less as measured with the same.

The pH of the first agent of the present disclosure at 20° C. is not particularly limited, but preferably ranges from pH 9 to 12.

The pH of the first agent of the present disclosure at 20° C. is measured by filling a sample bottle (Food 140: Manufactured by Daiichi Glass Co., Ltd.) with 120 g of the first agent prepared by the usual method, allowing it to stand at 20° C. for 24 hours, and then measuring the pH of the stock solution using a glass electrode hydrogen ion concentration indicator (F-71, manufactured by HORIBA, Ltd.).

The second agent of the present disclosure is described.

The second agent of the present disclosure contains nonionic surfactant (A) from the viewpoint of the simultaneous dispensability of the first agent and the second agent after long-term storage.

Examples of ingredient (A) above used in the second agent of the present disclosure include, but are not particularly limited to, POE alkyl ethers, POEPOP alkyl ethers, POE fatty acid esters, POE sorbitan fatty acid esters, POE castor oil, POE hardened castor oil, POE sorbitol tetra fatty acid esters, POE phytosterols, glycerol fatty acid esters, sorbitan fatty acid esters, polyglycerol fatty acid esters, sucrose fatty acid esters, alkyl polyglucosides, N-alkyldimethylamine oxides, and alkanolamides, and one or two or more types thereof may be contained.

Among the examples of ingredient (A) above used in the second agent of the present disclosure, POE alkyl ethers are preferred from the viewpoint of the simultaneous dispensability of the first agent and the second agent after long-term storage.

Examples of the POE alkyl ether as ingredient (A) used in the second agent of the present disclosure include, but are not particularly limited to, POE (2) lauryl ether, POE (4.2) lauryl ether, POE (9) lauryl ether, POE (21) lauryl ether, POE (25) lauryl ether, POE (2) cetyl ether, POE (5) cetyl ether, POE (5.5) cetyl ether, POE (7) cetyl ether, POE (8) cetyl ether, POE (10) cetyl ether, POE (13) cetyl ether, POE (15) cetyl ether, POE (20) cetyl ether, POE (23) cetyl ether, POE (25) cetyl ether, POE (30) cetyl ether, POE (40) cetyl ether, POE (2) stearyl ether, POE (4) stearyl ether, POE (11) stearyl ether, POE (15) stearyl ether, POE (20) stearyl ether, POE (30) stearyl ether, POE (40) stearyl ether, POE (2) oleyl ether, POE (7) oleyl ether, POE (10) oleyl ether, POE (15) oleyl ether, POE (20) oleyl ether, POE (50) oleyl ether, POE (5) behenyl ether, POE (10) behenyl ether, POE (20) behenyl ether, and POE (30) behenyl ether, and one or two or more types thereof may be contained. Of these, one or more types selected from POE (10) cetyl ether or POE (20) cetyl ether are preferred from the viewpoint of the simultaneous dispensability of the first agent and the second agent after long-term storage.

Two or more types of ingredient (A) above used in the second agent of the present disclosure are preferably contained from the viewpoint of the simultaneous dispensability of the first agent and the second agent after long-term storage, and more preferably POE (10) cetyl ether and POE (20) cetyl ether are used in combination.

The content of ingredient (A) above used in the second agent of the present disclosure is not particularly limited, but ranges from preferably 0.6% to 4.9%, and more preferably 1.3% to 4.9%. If the content of ingredient (A) above is less than 0.6%, the simultaneous dispensability of the first agent and the second agent may deteriorate after long-term storage. If the content of ingredient (A) above exceeds 4.9%, no further effect may be obtained.

The second agent of the hair dye composition in the present disclosure contains higher alcohol (B) having 12 to 24 carbon atoms from the viewpoint of the simultaneous dispensability and the mixability of the first agent and the second agent after long-term storage.

Examples of ingredient (B) above used in the second agent of the present disclosure include, but are not particularly limited to, lauryl alcohol, myristyl alcohol, cetanol, stearyl alcohol, cetostearyl alcohol, isostearyl alcohol, oleyl alcohol, arachyl alcohol, behenyl alcohol, octyldodecanol, and decyltetradecanol, and one or two or more types thereof may be contained.

Two or more types of ingredient (B) above used in the second agent of the present disclosure are preferably contained from the viewpoint of the simultaneous dispensability and the mixability of the first agent and the second agent after long-term storage, and more preferably cetanol and stearyl alcohol are used in combination.

The content of ingredient (B) above used in the second agent of the present disclosure is not particularly limited, but ranges from preferably 1.2% to 9.8%, and more preferably 2.5% to 7.4%. If the content of ingredient (B) above is less than 1.2% or more than 9.8%, the mixability of the first agent and the second agent may deteriorate after long-term storage.

The second agent of the present disclosure contains hydrocarbon (C) that is in a liquid or paste form at 25° C. from the viewpoint of the simultaneous dispensability and the mixability of the first agent and the second agent after long-term storage.

Among ingredient (C) above used in the second agent of the present disclosure, examples of the hydrocarbon that is in a liquid form at 25° C. include, but are not particularly limited to, liquid paraffin, liquid isoparaffin, light liquid isoparaffin, heavy liquid isoparaffin, squalane, and vegetable squalane, and examples of the hydrocarbon that is in a paste form at 25° C. include, but are not particularly limited to, vaseline, and one or two or more types thereof may be contained. Of these, liquid paraffin, liquid isoparaffin, and squalane are preferred from the viewpoint of the simultaneous dispensability of the first agent and the second agent after long-term storage, and liquid paraffin is more preferred from the viewpoint of the mixability of the first agent and the second agent after long-term storage.

The content of ingredient (C) above used in the second agent of the present disclosure is not particularly limited, but ranges from preferably 0.1% to 10%, more preferably 0.1% to 8%, and even more preferably 1% to 8% from the viewpoint of the simultaneous dispensability and the mixability of the first agent and the second agent after long-term storage. If the content of ingredient (C) above is less than 0.1%, the mixability of the first agent and the second agent may deteriorate after long-term storage. If the content of ingredient (C) above exceeds 10%, the simultaneous dispensability and the mixability of the first agent and the second agent may deteriorate after long-term storage.

The second agent of the present disclosure contains an oxidizing agent from the viewpoint of decolorizing properties.

Examples of the oxidizing agent used in the second agent of the present disclosure include, but are not particularly limited to, hydrogen peroxide, urea peroxide, sodium percarbonate, potassium percarbonate, ammonium persulfate, sodium peroxide, potassium peroxide, magnesium peroxide, barium peroxide, and calcium peroxide, and one or two or more types thereof may be contained.

The content of the oxidizing agent used in the second agent of the present disclosure is not particularly limited, but preferably ranges from 0.1% to 6%.

The viscosity of the second agent of the present disclosure at 20° C. is not particularly limited, but the viscosity preferably ranges from 100 mPa·s to 100,000 mPa·s.

The viscosity of the second agent of the present disclosure at 20° C. was measured by filling a sample bottle (Food 140: Manufactured by Dai-ichi Glass Co., Ltd.) with 120 g of the first agent prepared by the usual method and allowing it to stand at 20° C. for 24 hours, and then rotating it for 1 minute: at 60 rpm using an M4 rotor when the viscosity was 100 mPa·s or more and less than 10,000 mPa·s as measured with a B-type viscometer (model: Digital Viscometer TVB-10M, manufactured by Toki Sangyo Co., Ltd.); at 30 rpm using the M4 rotor when the viscosity was 10,000 mPa·s or more and less than 20,000 mPa·s as measured with the same; at 12 rpm using the M4 rotor when the viscosity was 20,000 mPa·s or more and less than 50,000 mPa·s as measured with the same; and at 6 rpm using the M4 rotor when the viscosity was 50,000 mPa·s or more and 100,000 mPa·s or less as measured with the same.

The pH of the second agent of the present disclosure at 20° C. is not particularly limited, but preferably ranges from pH 2 to 4.

The pH of the second agent of the present disclosure at 20° C. was measured by filling a sample bottle (Food 140: Manufactured by DAI-ICHI GLASS Co., Ltd.) with 120 g of the second agent prepared by the usual method, allowing it to stand at 20° C. for 24 hours, and then measuring the pH of the stock solution with a glass electrode hydrogen ion concentration indicator (F-71, manufactured by HORIBA, Ltd.).

The aerosol-type cream hair dye composition in the present disclosure may, if necessary, contain one or two or more types of various ingredients selected from a cationic surfactant, vegetable oil, ester oil, a fatty acid, a solid hydrocarbon, a wax, silicone, a water-soluble polymer, alcohols, a chelating agent, an antioxidant, a pH adjuster, a preservative, a moisturizer, and the like other than ingredient (A) above, ingredient (B) above and ingredient (C) above. Various ingredients other than ingredient (A) above, ingredient (B) above and ingredient (C) above may be contained in the first agent, the second agent, or both the first agent and the second agent of the aerosol-type cream hair dye composition in the present disclosure.

*The paragraphs are aligned in the order of the ingredients listed above.

The aerosol type cream hair dye composition in the present disclosure may contain a cationic surfactant from the viewpoint of excellent combing with fingers upon rinsing hair.

Examples of the cationic surfactant used in the aerosol type cream hair dye composition of the present disclosure include, but are not particularly limited to, lauryl trimethylammonium chloride, cetyltrimethylammonium chloride, stearyl trimethylammonium chloride, behenyl trimethylammonium chloride, and distearyldimethylammonium chloride, and one or two or more types thereof may be contained.

The content of the cationic surfactant used in the aerosol type cream hair dye composition of the present disclosure is not particularly limited, but preferably ranges from 0.1% to 10%.

The aerosol type cream hair dye composition in the present disclosure may contain water-soluble polymers from the viewpoint of excellent combing with fingers upon rinsing hair.

The aerosol type cream hair dye composition in the present disclosure may contain vegetable oil.

Examples of the vegetable oil used in the aerosol type cream hair dye composition of the present disclosure include, but are not particularly limited to, almond oil, avocado oil, argania spinosa kernel oil, Cananga odorata flower oil, olive oil, orange oil, apricot kernel oil, corn oil, sesame oil, pomegranate seed oil, shea butter, spearmint oil, damask rose flower oil, camellia oil, corn oil, Pelargonium graveolens flower oil, Nigella sativa seed oil, peppermint oil, Hypophaea rhamnoides seed oil, sunflower seed oil, grape seed oil, Plukenetia volubilis seed oil, Luffa cylindrica seed oil, bergamot fruit oil, jojoba oil, macadamia nut oil, mink oil, meadowfoam oil, peach kernel oil, coconut oil, eucalyptus oil, yucha oil, lavender oil, lemon fruit oil, rose hip oil, rosemary oil, and evening primrose oil, and one or two or more types thereof may be contained.

The content of the vegetable oil used in the aerosol type cream hair dye composition of the present disclosure is not particularly limited, but preferably ranges from 0.01% to 20%.

The aerosol type cream hair dye composition in the present disclosure may contain ester oil.

Examples of the ester oil used in the aerosol type cream hair dye composition of the present disclosure include, but are not particularly limited to, diisopropyl adipate, diisobutyl adipate, bis(2-ethylhexyl) adipate, isopropyl myristate, cetyl ethyl hexanoate, cetyl isooctanoate, isononyl isonononanoate, isodecyl isonononanoate, isotridecyl isonononanoate, diisopropyl sebacate, octyldodecyl myristate, isopropyl palmitate, hexyl laurate, oleyl oleate, decyl oleate, octyldodecyl oleate, oleyl erucate, octyldodecyl erucate, triisodecyl myristate, isostearyl myristate, 2-ethylhexyl palmitate, lauryl lactate, octyldodecyl lactate, isocetyl stearate, isocetyl isostearate, ethylene glycol di-2-ethylhexylate, glyceryl tricaprylate, and neopentyl glycol dicaprate, and one or two or more thereof may be contained.

The content of the ester oil used in the aerosol type cream hair dye composition of the present disclosure is not particularly limited, but preferably ranges from 0.1% to 20%.

The aerosol type cream hair dye composition in the present disclosure may contain a fatty acid.

Examples of the fatty acid used in the aerosol type cream hair dye composition of the present disclosure include, but are not particularly limited to, lauric acid, myristic acid, palmitic acid, stearic acid, behenic acid, isostearic acid, hydroxystearic acid, oleic acid, undecylenic acid, linoleic acid, ricinoleic acid, and lanolin fatty acid, and one or two or more types thereof may be contained.

The content of the fatty acid used in the aerosol type cream hair dye composition of the present disclosure is not particularly limited, but preferably ranges from 0.1% to 10%.

The aerosol type cream hair dye composition in the present disclosure may contain a solid hydrocarbon.

Examples of the solid hydrocarbon used in the aerosol type cream hair dye composition of the present disclosure include, but are not particularly limited to, microcrystalline wax, high melting point microcrystalline wax, synthetic hydrocarbon wax, polyethylene wax, and synthetic wax, and one or two or more types thereof may be contained.

The content of the solid hydrocarbon used in the aerosol type cream hair dye composition of the present disclosure is not particularly limited, but preferably ranges from 0.1% to 20%.

The aerosol type cream hair dye composition in the present disclosure may contain waxes.

Examples of the waxes used in the aerosol type cream hair dye composition of the present disclosure include, but are not particularly limited to, candelilla wax, rice bran wax, carnauba wax, bees wax, and sunflower seed wax, and one or two or more types thereof may be contained.

The content of the waxes used in the aerosol type cream hair dye composition of the present disclosure is not particularly limited, but preferably ranges from 0.1% to 20%.

The aerosol type cream hair dye composition in the present disclosure may contain silicone from the viewpoint of excellent combing of finished hair with fingers.

Examples of the silicone used in the aerosol type cream hair dye composition of the present disclosure include, but are not particularly limited to, decamethylcyclopentasiloxane, poly(oxyethylene·oxypropylene) methylpolysiloxane copolymer, polyoxyethylene·methylpolysiloxane copolymer, methylphenylpolysiloxane, methylpolysiloxane, and highly polymerized methylpolysiloxane, and one or two or more types thereof may be contained.

The content of the silicone used in the aerosol type cream hair dye composition of the present disclosure is not particularly limited, but preferably ranges from 0.1% to 20%.

Examples of the water-soluble polymer used in the aerosol type cream hair dye composition of the present disclosure include, but are not particularly limited to, acrylamide·acrylic acid·dimethyldiallylammonium chloride copolymer, dimethyldiallylammonium chloride·acrylamide copolymer, dimethyldiallylammonium chloride-acrylic acid copolymer, and poly(dimethyldimethylene pyrrolidinium chloride), and one or two or more types thereof may be contained.

The content of the water-soluble polymer used in the aerosol type cream hair dye composition of the present disclosure is not particularly limited, but preferably ranges from 0.01% to 5%.

The aerosol type cream hair dye composition in the present disclosure may contain alcohols from the viewpoint of hair dyeability.

Examples of the alcohols used in the aerosol type cream hair dye composition of the present disclosure include, but are not particularly limited to, ethanol, diethylene glycol monoethyl ether, glycerin, diglycerin, ethylene glycol, diethylene glycol, propylene glycol, dipropylene glycol, 1,3-butylene glycol, and polyethylene glycol, and one or two more types thereof may be contained.

The content of the alcohols used in the aerosol type cream hair dye composition of the present disclosure is not particularly limited, but preferably ranges from 0.1% to 10%.

The aerosol type cream hair dye composition in the present disclosure may contain a chelating agent.

Examples of the chelating agent used in the aerosol type cream hair dye composition of the present disclosure include, but are not particularly limited to, ethylenediaminetetraacetic acid tetrasodium salt tetrahydrate, pentasodium diethylenetriamine pentaacetate, and hydroxyethane diphosphonic acid, and one or two or more types thereof may be contained.

The content of the chelating agent used in the aerosol type cream hair dye composition of the present disclosure is not particularly limited, but preferably ranges from 0.01% to 1%.

The aerosol type cream hair dye composition in the present disclosure may contain an antioxidant.

Examples of the antioxidant used in the aerosol type cream hair dye composition of the present disclosure include, but are not particularly limited to, sodium sulfite, L-ascorbic acid, and dl-α-tocopherol, and one or two or more types thereof may be contained.

The content of the antioxidant used in the aerosol type cream hair dye composition of the present disclosure is not particularly limited, but preferably ranges from 0.1% to 5%.

The aerosol type cream hair dye composition in the present disclosure may contain a pH adjuster.

Examples of the pH adjuster used in the aerosol type cream hair dye composition of the present disclosure include, but are not particularly limited to, potassium hydroxide, sodium DL-malate, magnesium sulfate, citric acid, sodium citrate dihydrate, phosphoric acid, and sodium monohydrogen phosphate, and one or two or more types thereof may be contained.

The content of the pH adjuster used in the aerosol type cream hair dye composition of the present disclosure is not particularly limited, but preferably ranges from 0.01% to 5%.

The aerosol type cream hair dye composition in the present disclosure may contain a preservative.

Examples of the preservative used in the aerosol type cream hair dye composition of the present disclosure include, but are not particularly limited to, methyl paraoxibenzoate, propyl paraoxibenzoate, butyl paraoxibenzoate, phenoxyethanol, and ethylhexylglycerin, and one or two or more types thereof may be contained.

The content of the preservative used in the aerosol type cream hair dye composition of the present disclosure is not particularly limited, but preferably ranges from 0.01% to 1%.

The aerosol type cream hair dye composition in the present disclosure may contain a moisturizer.

Examples of the moisturizer used in the aerosol type cream hair dye composition of the present disclosure include, but are not particularly limited to, an arch choke leaf extract, an ai leaf/stem extract, a red clover flower extract, an akebia stem extract, an Angelica keiskei extract, an ivyleaf morning glory callus culture extract, an arnica extract, an aloe extract, a strawberry seed extract, an dill seed extract, a turmeric extract, an edelweiss extract, an Fomes officinalis (mushroom) extract, an Scutellaria baicalensis root extract, a Cladosiphon okamuramuis extract, a Panax ginseng root extract, a Lamium album extract, a Laminaria diabolica extract, a watercress extract, a Kjellmaniella crassifolia extract, a kamille extract, an Avena sativa (oat) kernel extract, an Artemisia capillaris extract, a licorice extract, a raspberry extract, a kiwi extract, a cucumber extract, a Sophora angustifolia root extract, an Alpinia speciosa leaf extract, a burdock root extract, a rice extract, a rice bran extract, an Eisenia arborea extract, a pomegranate fruit extract, a Prumus lannesiana flower extract, a common soapwort extract, a hawthorn extract, a Zanthoxylum piperitum peel extract, a shea butter extract, a Rehmannia glutinosa extract, a Perilla ocymoides leaf extract, a linden extract, a Filipendula extract, a peony extract, a ginger extract, a Tremella fuciformis (Mushroom) extract, a Caesalpinia sappan bark extract, an Equisetum arvense stem extract, a Porphyra yezoensis extract, an Urtica dioica leaf extract, an ivy extract, a Crataegus oxyacantha fruit extract, a Sambucus nigra extract, an Achillea millefolium flower extract, a Mentha piperrita extract, a sage extract, a Malva sylvestris (Mallow) flower extract, a Cnidium rhizome extract, a Swertia japonica extract, a jujube extract, a thyme extract, a Rosa damascena extract, a green tea extract, a Eugenia caryophyllus (clove) flower extract, a Citrus unshiu peel extract, a (Camellia japonica flower extract, an Angelica acutiloba root extract, a Eucheuma serra/Grateloupia sparsa/Saccharina angustata/Ulva linza/Undaria pinnatifida extract, a tomato extract, a carrot extract, a garlic extract, a Nymphaea caerulea flower extract, a Rosa canina extract, a Nelumbium speciosum flower extract, a honey extract, a Hamamelis virginiana extract, a Parietaria officinalis extract, a beer extract, a Fucus vesiculosus extract, a sunflower seed extract, a Geranium robertiamum extract, a Portulaca pilosa extract, a Vaccinium myrtillus extract, a Tussilago farfara leaf extract, a propolis extract, a pine extract, an Origamim majorana leaf extract, a marronnier extract, a mango fruit extract, a mangosteen pericarp extract, a Microcitrus australasica fruit extract, a Menyanthes trifoliata leaf extract, an evening primrose seed extract, a Terminalia catappa leaf extract, a peach fruit extract, peach fruit water, a peach leaf extract, a Saxifraga sarmentosa extract, a Citrus junos extract, a European birch leaf extract, a (Coix lacryma-jobi ma-yuen seed extract, a peanut seedcoat extract, a Nephelium longana seed extract, an apple extract, a lettuce extract, a lemon grass leaf/stem extract, a rosemary extract, an Anthemis nobilis extract, a royal jelly extract, a logwood extract, a hydrolyzed okra seed extract, a hydrolyzed silk liquid, a hydrolyzed soybean extract, a seaweed extract, a brown algae extract, a black tea extract, a yeast extract, an Oryza sativa (rice) lees extract, a soy protein hydrolysate, collagen, and hyaluronic acid, and one or two or more types thereof may be contained.

The content of the moisturizer used in the aerosol type cream hair dye composition of the present disclosure is not particularly limited, but preferably ranges from 0.01% to 10%.

The viscosity of the aerosol type cream hair dye composition at 20° C. in the present disclosure is not particularly limited, but preferably ranges from 100 mPa·s to 100,000 mPa·s from the viewpoint of applicability.

The viscosity of the aerosol type cream hair dye composition of the present disclosure at 20° C. was measured by mixing the first agent and the second agent, filling a sample bottle (Food 140: Manufactured by DAI-ICHI GLASS Co., Ltd.) with 120 g of the mixture, adjusting the temperature to 20° C., and then rotating it for 1 minute: at 60 rpm using an M4 rotor when the viscosity was 100 mPa·s or more and less than 10,000 mPa·s as measured with a B-type viscometer (Model: Digital Viscometer TVB-10M, manufactured by Toki Sangyo Co., Ltd); at 30 rpm using the M4 rotor when the viscosity was 10,000 mPa·s or more and less than 20,000 mPa·s as measured with the same; at 12 rpm using the M4 rotor when the viscosity was 20,000 mPa·s or more and less than 50,000 mPa·s as measured with the same; or at 6 rpm using the M4 rotor when the viscosity was 50,000 mPa·s or more and 100,000 mPa·s or less as measured with the same.

The pH of the aerosol type cream hair dye composition at 20° C. in the present disclosure is not particularly limited, but preferably ranges from pH 8 to 12 from the viewpoint of hair dyeability.

The pH of the aerosol type cream hair dye composition of the present disclosure at 20° C. was measured by mixing the first agent and the second agent, filling a sample bottle (Food 140: manufactured by DAI-ICHI GLASS Co., Ltd.) with 120 g of the mixture, adjusting the temperature to 20° C., and then measuring the pH with a glass electrode hydrogen ion concentration indicator (F-71, manufactured by the HORIBA, Ltd.).

The container of the aerosol type cream hair dye composition of the present disclosure is structured such that inner bags attached to the aerosol container are filled with the first agent and the second agent, respectively, and then the internal spaces of the aerosol container and the inner bags are further filled with compressed gas. Examples of the compressed gas include, but are not particularly limited to, carbon dioxide, nitrogen, and nitrous oxide, and one or two or more types thereof may be used for filling the spaces.

Examples of the method for mixing the first agent and the second agent of the aerosol type cream hair dye composition of the present disclosure include, but are not particularly limited to, a method that involves mixing the first agent and the second agent on a tray, a method that involves mixing the first agent and the second agent on the palm of a hand wearing a glove, and a method that involves dispensing the first agent and the second agent on a brush, and applying them while mixing them.

Examples of a tool for mixing the first agent and the second agent of the aerosol type cream hair dye composition of the present disclosure include, but are not particularly limited to, a brush, a comb, a brush (hake), and a spatula.

Examples of a method for applying the aerosol type cream hair dye composition of the present disclosure include, but are not particularly limited to, a method that involves mixing the first agent and the second agent dispensed while directly applying them to the hair, a method that involves applying the prepared hair dye composition by hand to the hair, and a method that involves applying the prepared hair dye composition on a brush and then applying the composition to the hair.

### EXAMPLES

The present disclosure is described in more detail below with examples, which do not limit the present disclosure in any way.

An evaluation test presented herein was conducted by varying the ingredients contained in the first agent and the second agent of the hair dye composition, as well as their contents. All units indicating the content of each ingredient are all mass %, and each composition was prepared by a usual method.

<Simultaneous Dispensability of the First Agent and the Second Agent after Long-Term Storage>

In the evaluation test concerning “simultaneous dispensability of the first agent and the second agent after long-term storage” described herein, the inner bags of an aerosol container were filled with 40 g of the first agent and 40 g of the second agent, respectively, which had been prepared by the usual method, the internal spaces of the aerosol container and the inner bags were filled with nitrogen gas, and then the pressure was adjusted to 0.55 Mpa to prepare each evaluation sample. The evaluation sample was stored under 40° C. conditions for 6 months, and then left to stand at 20° C. for 1 day to adjust the temperature. After that, using a mechanism for simultaneously dispensing the first agent and the second agent, the dispensed amounts of the first agent and the second agent were measured at the point when 30 g of either one agent had been dispensed first, so as to obtain the ratio of the dispensed amount of the second agent to that of the first agent. The evaluation criteria are as follows.

<Evaluation Criteria for Simultaneous Dispensability of the First Agent and the Second Agent after Long-Term Storage>
A: The ratio of the amount of the second agent dispensed to that of the first agent dispensed at the point when 30 g of either one agent is dispensed first falls within the range of 0.8 to 1.2.
B: The ratio of the amount of the second agent dispensed to that of the first agent dispensed at the point when 30 g of either one agent is dispensed first falls within the range of 0.7 to 1.5.
NG: Due to separation immediately after preparation of the product, no evaluation could be made.
   <Mixability of the First Agent and the Second Agent after Long-Term Storage>

In the evaluation test concerning “Mixability of the first agent and the second agent after long-term storage” described herein, the inner bags of an aerosol container were filled with 40 g of the first agent and 40 g of the second agent, respectively, which had been prepared by the usual method, the internal spaces of the aerosol container and the inner bags were filled with nitrogen gas, and then the pressure was adjusted to 0.55 Mpa to prepare each evaluation sample. The evaluation sample was stored for six months under 40° C. conditions, left to stand at 20° C. for one day to adjust the temperature. Then, 30 g each of the sample was weighed in a 200-mL Griffin beaker. The beaker was tilted at a 30-degree angle from the vertical position and stirred in a circular motion with a glass rod. The evaluation was made as described below based on the number of stirring the mixture until it was uniformly mixed.

<Evaluation Method for Mixability of the First Agent and the Second Agent after Long-Term Storage>
A: Mixed uniformly by less than 50 times of stirring.
B: Mixed uniformly by less than 100 times of stirring.
C: Not uniformly mixed even after 100 times of stirring, and lumps and remaining mixtures were confirmed.
NG: Due to separation immediately after preparation of the first agent or the second agent, no evaluation could be made.

### (First Evaluation Test)

In the first evaluation test, evaluation was made by varying the content and the type of ingredient (A) in the first agent of the hair dye composition prepared by a usual method. Table 1 lists the ingredients, contents and evaluation results of the first agent and the second agent.

TABLE 1

| | | Example | Example | Example | Example | Example | Example | Comparative |
|---|---|---|---|---|---|---|---|---|
| | | 1 | 2 | 3 | 4 | 5 | 6 | example 1 |
| Ingredient name (First agent) | | | | | | | | |
| (A) | Polyoxyethylene (20) stearyl ether | 0.70 | 1.40 | 2.70 | 5.40 | — | 4.00 | — |
| (A) | Polyoxyethylene (5) behenyl ether | 0.30 | 0.70 | 1.30 | 2.60 | 4.00 | — | — |
| (B) | Cetanol | 0.10 | 0.10 | 0.10 | 0.10 | 0.10 | 0.10 | 0.10 |
| (B) | Cetostearyl alcohol | 2.50 | 2.50 | 2.50 | 2.50 | 2.50 | 2.50 | 2.50 |
| (C) | Liquid paraffin | 5.00 | 5.00 | 5.00 | 5.00 | 5.00 | 5.00 | 5.00 |
| | Monoethanolamine | 4.80 | 4.80 | 4.80 | 4.80 | 4.80 | 4.80 | 4.80 |
| | Para-aminophenol | 0.10 | 0.10 | 0.10 | 0.10 | 0.10 | 0.10 | 0.10 |
| | Paraphenylenediamine | 2.00 | 2.00 | 2.00 | 2.00 | 2.00 | 2.00 | 2.00 |
| | Meta-aminophenol | 0.20 | 0.20 | 0.20 | 0.20 | 0.20 | 0.20 | 0.20 |
| | Meta-phenylenediamine sulfate | 0.30 | 0.30 | 0.30 | 0.30 | 0.30 | 0.30 | 0.30 |
| | Resorcin | 0.50 | 0.50 | 0.50 | 0.50 | 0.50 | 0.50 | 0.50 |
| | Stearyltrimethylammonium chloride | 0.70 | 0.70 | 0.70 | 0.70 | 0.70 | 0.70 | 0.70 |
| | Stearic acid | 0.50 | 0.50 | 0.50 | 0.50 | 0.50 | 0.50 | 0.50 |
| | Poly N,N-dimethyl-3,4-dimethylene- | 0.42 | 0.42 | 0.42 | 0.42 | 0.42 | 0.42 | 0.42 |
| | pyrrolidinium chloride | | | | | | | |
| | Pentasodium diethylenetriamine | 0.92 | 0.92 | 0.92 | 0.92 | 0.92 | 0.92 | 0.92 |
| | pentaacetate | | | | | | | |
| | Sodium sulfite | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 |
| | Magnesium sulfate | 1.00 | 1.00 | 1.00 | 1.00 | 1.00 | 1.00 | 1.00 |
| | DL-sodium malate | 1.00 | 1.00 | 1.00 | 1.00 | 1.00 | 1.00 | 1.00 |
| | Purified water | 78.26 | 77.16 | 75.26 | 71.26 | 75.26 | 75.26 | 79.26 |
| | Total | 100.00 | 100.00 | 100.00 | 100.00 | 100.00 | 100.00 | 100.00 |
| Ingredient name (Second agent) | | | | | | | | |
| (A) | Polyoxyethylene (10) cetyl ether | 1.00 | 1.00 | 1.00 | 1.00 | 1.00 | 1.00 | 1.00 |
| (A) | Polyoxyethylene (20) cetyl ether | 1.50 | 1.50 | 1.50 | 1.50 | 1.50 | 1.50 | 1.50 |
| (B) | Cetanol | 3.70 | 3.70 | 3.70 | 3.70 | 3.70 | 3.70 | 3.70 |
| (B) | Stearyl alcohol | 1.20 | 1.20 | 1.20 | 1.20 | 1.20 | 1.20 | 1.20 |
| (C) | Liquid paraffin | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 |
| | Hydroxyethane diphosphonic acid | 0.08 | 0.08 | 0.08 | 0.08 | 0.08 | 0.08 | 0.08 |
| | Phosphoric acid | 0.02 | 0.02 | 0.02 | 0.02 | 0.02 | 0.02 | 0.02 |
| | Sodium monohydrogen phosphate | 0.10 | 0.10 | 0.10 | 0.10 | 0.10 | 0.10 | 0.10 |
| | Purified water | 83.09 | 83.09 | 83.09 | 83.09 | 83.09 | 83.09 | 83.09 |
| | Total | 100.00 | 100.00 | 100.00 | 100.00 | 100.00 | 100.00 | 100.00 |
| Evaluation | Simultaneous dispensability of the | B | A | A | A | B | B | NG |
| items | first agent and the second agent | | | | | | | |
| | Mixability of the first agent | A | A | A | A | A | A | NG |
| | and the second agent | | | | | | | |

From Examples 1 to 6 shown in Table 1, good results were obtained for the simultaneous dispensability and the mixability of the first agent and the second agent after long-term storage.

### (Second Evaluation Test)

In the second evaluation test, evaluation was made by varying the content and the type of ingredient (B) in the first agent of the hair dye composition prepared by the usual method. Table 2 lists the ingredients, contents and evaluation results of the first agent and the second agent.

TABLE 2

| | | Example | Example | Example | Example | Example | Comparative |
|---|---|---|---|---|---|---|---|
| | | 7 | 8 | 9 | 10 | 11 | example 2 |
| Ingredient name (First agent) | | | | | | | |
| (A) | Polyoxyethylene (20) stearyl ether | 2.70 | 2.70 | 2.70 | 2.70 | 2.70 | 2.70 |
| (A) | Polyoxyethylene (5) behenyl ether | 1.30 | 1.30 | 1.30 | 1.30 | 1.30 | 1.30 |
| (B) | Cetanol | 0.02 | 0.05 | 0.20 | 0.40 | 2.60 | — |
| (B) | Cetostearyl alcohol | 0.48 | 1.25 | 3.80 | 5.10 | — | — |
| (C) | Liquid paraffin | 5.00 | 5.00 | 5.00 | 5.00 | 5.00 | 5.00 |
| | Monoethanolamine | 4.80 | 4.80 | 4.80 | 4.80 | 4.80 | 4.80 |
| | Para-aminophenol | 0.10 | 0.10 | 0.10 | 0.10 | 0.10 | 0.10 |
| | Paraphenylenediamine | 2.00 | 2.00 | 2.00 | 2.00 | 2.00 | 2.00 |
| | Meta-aminophenol | 0.20 | 0.20 | 0.20 | 0.20 | 0.20 | 0.20 |
| | Meta-phenylenediamine sulfate | 0.30 | 0.30 | 0.30 | 0.30 | 0.30 | 0.30 |
| | Resorcin | 0.50 | 0.50 | 0.50 | 0.50 | 0.50 | 0.50 |
| | Stearyltrimethylammonium chloride | 0.70 | 0.70 | 0.70 | 0.70 | 0.70 | 0.70 |
| | Stearic acid | 0.50 | 0.50 | 0.50 | 0.50 | 0.50 | 0.50 |
| | Poly N,N-dimethyl-3,4-dimethylene- | 0.42 | 0.42 | 0.42 | 0.42 | 0.42 | 0.42 |
| | pyrrolidinium chloride | | | | | | |
| | Pentasodium diethylenetriamine | 0.92 | 0.92 | 0.92 | 0.92 | 0.92 | 0.92 |
| | pentaacetate | | | | | | |
| | Sodium sulfite | 0.20 | 0.20 | 0.20 | 0.20 | 0.20 | 0.20 |
| | L-ascorbic acid | 0.50 | 0.50 | 0.50 | 0.50 | 0.50 | 0.50 |
| | Magnesium sulfate | 1.00 | 1.00 | 1.00 | 1.00 | 1.00 | 1.00 |
| | DL-sodium malate | 1.00 | 1.00 | 1.00 | 1.00 | 1.00 | 1.00 |
| | Purified water | 77.36 | 76.56 | 73.86 | 72.36 | 75.26 | 77.86 |
| | Total | 100.00 | 100.00 | 100.00 | 100.00 | 100.00 | 100.00 |
| Ingredient name (Second agent) | | | | | | | |
| (A) | Polyoxyethylene (10) cetyl ether | 1.00 | 1.00 | 1.00 | 1.00 | 1.00 | 1.00 |
| (A) | Polyoxyethylene (20) cetyl ether | 1.50 | 1.50 | 1.50 | 1.50 | 1.50 | 1.50 |
| (B) | Cetanol | 3.70 | 3.70 | 3.70 | 3.70 | 3.70 | 3.70 |
| (B) | Stearyl alcohol | 1.20 | 1.20 | 1.20 | 1.20 | 1.20 | 1.20 |
| (C) | Liquid paraffin | 2.00 | 2.00 | 2.00 | 2.00 | 2.00 | 2.00 |
| | Hydrogen peroxide | 5.81 | 5.81 | 5.81 | 5.81 | 5.81 | 5.81 |
| | Polyethylene glycol 400 | 1.50 | 1.50 | 1.50 | 1.50 | 1.50 | 1.50 |
| | Hydroxyethane diphosphonic acid | 0.08 | 0.08 | 0.08 | 0.08 | 0.08 | 0.08 |
| | Phosphoric acid | 0.02 | 0.02 | 0.02 | 0.02 | 0.02 | 0.02 |
| | Sodium monohydrogen phosphate | 0.10 | 0.10 | 0.10 | 0.10 | 0.10 | 0.10 |
| | Purified water | 83.09 | 83.09 | 83.09 | 83.09 | 83.09 | 83.09 |
| | Total | 100.00 | 100.00 | 100.00 | 100.00 | 100.00 | 100.00 |
| Evaluation | Simultaneous dispensability of the | A | A | A | A | B | NG |
| items | first agent and the second agent | | | | | | |
| | Mixability of the first agent and the second | B | A | A | B | A | NG |
| | agent after 6 months of storage at 40° C. | | | | | | |

From Examples 7 to 11 shown in Table 2, good results were obtained for the simultaneous dispensability and the mixability of the first agent and the second agent after long-term storage.

### (Third Evaluation Test)

In the third evaluation test, evaluation was made by varying the content and the type of ingredient (C) in the first agent of the hair dye composition prepared by the usual method. Table 3 lists the ingredients, contents and evaluation results of the first gent and the second agent.

TABLE 3

| | | Example | Example | Example | Example | Example | Example | Example | Comparative |
|---|---|---|---|---|---|---|---|---|---|
| | | 12 | 13 | 14 | 15 | 16 | 17 | 18 | example 3 |
| Ingredient name (First agent) | | | | | | | | | |
| (A) | Polyoxyethylene (20) stearyl ether | 2.70 | 2.70 | 2.70 | 2.70 | 2.70 | 2.70 | 2.70 | 2.70 |
| (A) | Polyoxyethylene (5) behenyl ether | 1.30 | 1.30 | 1.30 | 1.30 | 1.30 | 1.30 | 1.30 | 1.30 |
| (B) | Cetanol | 0.10 | 0.10 | 0.10 | 0.10 | 0.10 | 0.10 | 0.10 | 0.10 |
| (B) | Cetostearyl alcohol | 2.50 | 2.50 | 2.50 | 2.50 | 2.50 | 2.50 | 2.50 | 2.50 |
| (C) | Liquid paraffin | 0.10 | 1.00 | 10.00 | 15.00 | — | — | — | — |
| (C) | Liquid isoparaffin | — | — | — | — | 5.00 | — | — | — |
| (C) | Squalane | — | — | — | — | — | 5.00 | — | — |
| (C) | Vaseline | — | — | — | — | — | — | 5.00 | — |
| | Monoethanolamine | 4.80 | 4.80 | 4.80 | 4.80 | 4.80 | 4.80 | 4.80 | 4.80 |
| | Para-aminophenol | 0.10 | 0.10 | 0.10 | 0.10 | 0.10 | 0.10 | 0.10 | 0.10 |
| | Paraphenylenediamine | 2.00 | 2.00 | 2.00 | 2.00 | 2.00 | 2.00 | 2.00 | 2.00 |
| | Meta-aminophenol | 0.20 | 0.20 | 0.20 | 0.20 | 0.20 | 0.20 | 0.20 | 0.20 |
| | Meta-phenylenediamine sulfate | 0.30 | 0.30 | 0.30 | 0.30 | 0.30 | 0.30 | 0.30 | 0.30 |
| | Resorcin | 0.50 | 0.50 | 0.50 | 0.50 | 0.50 | 0.50 | 0.50 | 0.50 |
| | Stearyltrimethylammonium chloride | 0.70 | 0.70 | 0.70 | 0.70 | 0.70 | 0.70 | 0.70 | 0.70 |
| | Stearic acid | 0.50 | 0.50 | 0.50 | 0.50 | 0.50 | 0.50 | 0.50 | 0.50 |
| | Poly N,N-dimethyl-3,4-dimethylene- | 0.42 | 0.42 | 0.42 | 0.42 | 0.42 | 0.42 | 0.42 | 0.42 |
| | pyrrolidinium chloride | | | | | | | | |
| | Pentasodium diethylenetriamine | 0.92 | 0.92 | 0.92 | 0.92 | 0.92 | 0.92 | 0.92 | 0.92 |
| | pentaacetate | | | | | | | | |
| | Sodium sulfite | 0.20 | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 | 0.50 |
| | Magnesium sulfate | 1.00 | 1.00 | 1.00 | 1.00 | 1.00 | 1.00 | 1.00 | 1.00 |
| | DL-sodium malate | 1.00 | 1.00 | 1.00 | 1.00 | 1.00 | 1.00 | 1.00 | 1.00 |
| | Purified water | 80.16 | 79.26 | 70.26 | 65.26 | 75.26 | 75.26 | 75.26 | 80.26 |
| | Total | 100.00 | 100.00 | 100.00 | 100.00 | 100.00 | 100.00 | 100.00 | 100.00 |
| Ingredient name (Second agent) | | | | | | | | | |
| (A) | Polyoxyethylene (10) cetyl ether | 1.00 | 1.00 | 1.00 | 1.00 | 1.00 | 1.00 | 1.00 | 1.00 |
| (A) | Polyoxyethylene (20) cetyl ether | 1.50 | 1.50 | 1.50 | 1.50 | 1.50 | 1.50 | 1.50 | 1.50 |
| (B) | Cetanol | 3.70 | 3.70 | 3.70 | 3.70 | 3.70 | 3.70 | 3.70 | 3.70 |
| (B) | Stearyl alcohol | 1.20 | 1.20 | 1.20 | 1.20 | 1.20 | 1.20 | 1.20 | 1.20 |
| (C) | Liquid paraffin | 2.00 | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 | 1.50 |
| | Hydroxyethane diphosphonic acid | 0.08 | 0.08 | 0.08 | 0.08 | 0.08 | 0.08 | 0.08 | 0.08 |
| | Phosphoric acid | 0.02 | 0.02 | 0.02 | 0.02 | 0.02 | 0.02 | 0.02 | 0.02 |
| | Sodium monohydrogen phosphate | 0.10 | 0.10 | 0.10 | 0.10 | 0.10 | 0.10 | 0.10 | 0.10 |
| | Purified water | 83.09 | 83.09 | 83.09 | 83.09 | 83.09 | 83.09 | 83.09 | 83.09 |
| | Total | 100.00 | 100.00 | 100.00 | 100.00 | 100.00 | 100.00 | 100.00 | 100.00 |
| Evaluation | Simultaneous dispensability of the | A | A | A | B | A | A | B | A |
| items | first agent and the second agent | | | | | | | | |
| | Mixability of the first agent | B | A | A | B | B | B | B | C |
| | and the second agent | | | | | | | | |

From Examples 12 to 18 shown in Table 3, good results were obtained for the term storage.

### (Fourth Evaluation Test)

In the fourth evaluation test, evaluation was made by varying the content and the type of ingredient (A) in the second agent of the hair dye composition prepared by the usual method. Table 4 lists the ingredients, contents and evaluation results of the first agent and the second agent.

TABLE 4

| | | Example | Example | Example | Example | Example | Comparative |
|---|---|---|---|---|---|---|---|
| | | 19 | 20 | 21 | 22 | 23 | example 4 |
| Ingredient name (First agent) | | | | | | | |
| (A) | Polyoxyethylene (20) stearyl ether | 2.70 | 2.70 | 2.70 | 2.70 | 2.70 | 2.70 |
| (A) | Polyoxyethylene (5) behenyl ether | 1.30 | 1.30 | 1.30 | 1.30 | 1.30 | 1.30 |
| (B) | Cetanol | 0.10 | 0.10 | 0.10 | 0.10 | 0.10 | 0.10 |
| (B) | Cetostearyl alcohol | 2.50 | 2.50 | 2.50 | 2.50 | 2.50 | 2.50 |
| (C) | Liquid paraffin | 5.00 | 5.00 | 5.00 | 5.00 | 5.00 | 5.00 |
| | Monoethanolamine | 4.80 | 4.80 | 4.80 | 4.80 | 4.80 | 4.80 |
| | Para-aminophenol | 0.10 | 0.10 | 0.10 | 0.10 | 0.10 | 0.10 |
| | Paraphenylenediamine | 2.00 | 2.00 | 2.00 | 2.00 | 2.00 | 2.00 |
| | Meta-aminophenol | 0.20 | 0.20 | 0.20 | 0.20 | 0.20 | 0.20 |
| | Meta-phenylenediamine sulfate | 0.30 | 0.30 | 0.30 | 0.30 | 0.30 | 0.30 |
| | Resorcin | 0.50 | 0.50 | 0.50 | 0.50 | 0.50 | 0.50 |
| | Stearyltrimethylammonium chloride | 0.70 | 0.70 | 0.70 | 0.70 | 0.70 | 0.70 |
| | Stearic acid | 0.50 | 0.50 | 0.50 | 0.50 | 0.50 | 0.50 |
| | Poly N,N-dimethyl-3,4-dimethylene- | 0.42 | 0.42 | 0.42 | 0.42 | 0.42 | 0.42 |
| | ovrrolidinium chloride | | | | | | |
| | Pentasodium diethylenetriamine | 0.92 | 0.92 | 0.92 | 0.92 | 0.92 | 0.92 |
| | pentaacetate | | | | | | |
| | Sodium sulfite | 0.20 | 0.20 | 0.20 | 0.20 | 0.20 | 0.20 |
| | L-ascorbic acid | 0.50 | 0.50 | 0.50 | 0.50 | 0.50 | 0.50 |
| | Magnesium sulfate | 1.00 | 1.00 | 1.00 | 1.00 | 1.00 | 1.00 |
| | DL-sodium malate | 1.00 | 1.00 | 1.00 | 1.00 | 1.00 | 1.00 |
| | Purified water | 75.26 | 75.26 | 75.26 | 75.26 | 75.26 | 75.26 |
| | Total | 100.00 | 100.00 | 100.00 | 100.00 | 100.00 | 100.00 |
| Ingredient name (Second agent) | | | | | | | |
| (A) | Polyoxyethylene (10) cetyl ether | 0.20 | 0.50 | 1.90 | 2.50 | — | — |
| (A) | Polyoxyethylene (20) cetyl ether | 0.40 | 0.80 | 3.00 | — | 2.50 | — |
| (B) | Cetanol | 3.70 | 3.70 | 3.70 | 3.70 | 3.70 | 3.70 |
| (B) | Stearyl alcohol | 1.20 | 1.20 | 1.20 | 1.20 | 1.20 | 1.20 |
| (C) | Liquid paraffin | 2.00 | 2.00 | 2.00 | 2.00 | 2.00 | 2.00 |
| | Hydrogen peroxide | 5.81 | 5.81 | 5.81 | 5.81 | 5.81 | 5.81 |
| | Polyethylene glycol 400 | 1.50 | 1.50 | 1.50 | 1.50 | 1.50 | 1.50 |
| | Hydroxyethane diphosphonic acid | 0.08 | 0.08 | 0.08 | 0.08 | 0.08 | 0.08 |
| | Phosphoric acid | 0.02 | 0.02 | 0.02 | 0.02 | 0.02 | 0.02 |
| | Sodium monohydrogen phosphate | 0.10 | 0.10 | 0.10 | 0.10 | 0.10 | 0.10 |
| | Purified water | 84.99 | 84.29 | 80.69 | 83.09 | 83.09 | 85.59 |
| | Total | 100.00 | 100.00 | 100.00 | 100.00 | 100.00 | 100.00 |
| Evaluation | Simultaneous dispensability of the | B | A | A | B | B | NG |
| items | first agent and the second agent | | | | | | |
| | Mixability of the first agent | A | A | A | A | A | NG |
| | and the second agent | | | | | | |

From Examples 19 to 23 shown in Table 4, good results were obtained for the term storage.

### (Fifth Evaluation Test)

In the fifth evaluation test, evaluation was made by varying the content and the type of ingredient (B) in the second agent of the hair dye composition prepared by the usual method. Table 5 lists the ingredients, contents and evaluation results of the first agent and the second agent.

TABLE 5

| | | Example | Example | Example | Example | Example | Example | Comparative |
|---|---|---|---|---|---|---|---|---|
| | | 24 | 25 | 26 | 27 | 28 | 29 | example 5 |
| Ingredient name (First agent) | | | | | | | | |
| (A) | Polyoxyethylene (20) stearyl ether | 2.70 | 2.70 | 2.70 | 2.70 | 2.70 | 2.70 | 2.70 |
| (A) | Polyoxyethylene (5) behenyl ether | 1.30 | 1.30 | 1.30 | 1.30 | 1.30 | 1.30 | 1.30 |
| (B) | Cetanol | 0.10 | 0.10 | 0.10 | 0.10 | 0.10 | 0.10 | 0.10 |
| (B) | Cetostearyl alcohol | 2.50 | 2.50 | 2.50 | 2.50 | 2.50 | 2.50 | 2.50 |
| (C) | Liquid paraffin | 5.00 | 5.00 | 5.00 | 5.00 | 5.00 | 5.00 | 5.00 |
| | Monoethanolamine | 4.80 | 4.80 | 4.80 | 4.80 | 4.80 | 4.80 | 4.80 |
| | Para-aminophenol | 0.10 | 0.10 | 0.10 | 0.10 | 0.10 | 0.10 | 0.10 |
| | Paraphenylenediamine | 2.00 | 2.00 | 2.00 | 2.00 | 2.00 | 2.00 | 2.00 |
| | Meta-aminophenol | 0.20 | 0.20 | 0.20 | 0.20 | 0.20 | 0.20 | 0.20 |
| | Meta-phenylenediamine sulfate | 0.30 | 0.30 | 0.30 | 0.30 | 0.30 | 0.30 | 0.30 |
| | Resorcin | 0.50 | 0.50 | 0.50 | 0.50 | 0.50 | 0.50 | 0.50 |
| | Stearyltrimethylammonium chloride | 0.70 | 0.70 | 0.70 | 0.70 | 0.70 | 0.70 | 0.70 |
| | Stearic acid | 0.50 | 0.50 | 0.50 | 0.50 | 0.50 | 0.50 | 0.50 |
| | Poly N,N-dimethyl-3,4-dimethylene- | 0.42 | 0.42 | 0.42 | 0.42 | 0.42 | 0.42 | 0.42 |
| | pyrrolidinium chloride | | | | | | | |
| | Pentasodium diethylenetriamine | 0.92 | 0.92 | 0.92 | 0.92 | 0.92 | 0.92 | 0.92 |
| | pentaacetate | | | | | | | |
| | Sodium sulfite | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 |
| | Magnesium sulfate | 1.00 | 1.00 | 1.00 | 1.00 | 1.00 | 1.00 | 1.00 |
| | DL-sodium malate | 1.00 | 1.00 | 1.00 | 1.00 | 1.00 | 1.00 | 1.00 |
| | Purified water | 75.26 | 75.26 | 75.26 | 75.26 | 75.26 | 75.26 | 75.26 |
| | Total | 100.00 | 100.00 | 100.00 | 100.00 | 100.00 | 100.00 | 100.00 |
| Ingredient name (Second agent) | | | | | | | | |
| (A) | Polyoxyethylene (10) cetyl ether | 1.00 | 1.00 | 1.00 | 1.00 | 1.00 | 1.00 | 1.00 |
| (A) | Polyoxyethylene (20) cetyl ether | 1.50 | 1.50 | 1.50 | 1.50 | 1.50 | 1.50 | 1.50 |
| (B) | Cetanol | 0.90 | 1.90 | 5.60 | 7.40 | 4.90 | — | — |
| (B) | Stearyl alcohol | 0.30 | 0.60 | 1.80 | 2.40 | — | 4.90 | — |
| (C) | Liquid paraffin | 2.00 | 2.00 | 2.00 | 2.00 | 2.00 | 2.00 | 2.00 |
| | Hydrogen peroxide | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 |
| | Hydroxyethane diphosphonic acid | 0.08 | 0.08 | 0.08 | 0.08 | 0.08 | 0.08 | 0.08 |
| | Phosphoric acid | 0.02 | 0.02 | 0.02 | 0.02 | 0.02 | 0.02 | 0.02 |
| | Sodium monohydrogen phosphate | 0.10 | 0.10 | 0.10 | 0.10 | 0.10 | 0.10 | 0.10 |
| | Purified water | 86.79 | 85.49 | 80.59 | 78.19 | 83.09 | 83.09 | 87.99 |
| | Total | 100.00 | 100.00 | 100.00 | 100.00 | 100.00 | 100.00 | 100.00 |
| Evaluation | Simultaneous dispensability of the | A | A | A | A | B | B | NG |
| items | first agent and the second agent | | | | | | | |
| | Mixability of the first agent | B | A | A | B | B | B | NG |
| | and the second agent | | | | | | | |

From Examples 24 to 29 shown in Table 5, good results were obtained for the term storage.

### (Sixth Evaluation Test)

In the sixth evaluation test, evaluation was made by varying the content and the type of ingredient (C) in the second agent of the hair dye composition prepared by the usual method. Table 6 lists the ingredients, contents and evaluation results of the first agent and the second agent.

TABLE 6

| | | Example | Example | Example | Example | Example | Example | Example | Comparative |
|---|---|---|---|---|---|---|---|---|---|
| | | 30 | 31 | 32 | 33 | 34 | 35 | 36 | example 6 |
| Ingredient name (First agent) | | | | | | | | | |
| (A) | Polyoxyethylene (20) stearyl ether | 2.70 | 2.70 | 2.70 | 2.70 | 2.70 | 2.70 | 2.70 | 2.70 |
| (A) | Polyoxyethylene (5) behenyl ether | 1.30 | 1.30 | 1.30 | 1.30 | 1.30 | 1.30 | 1.30 | 1.30 |
| (B) | Cetanol | 0.10 | 0.10 | 0.10 | 0.10 | 0.10 | 0.10 | 0.10 | 0.10 |
| (B) | Cetostearyl alcohol | 2.50 | 2.50 | 2.50 | 2.50 | 2.50 | 2.50 | 2.50 | 2.50 |
| (C) | Liquid paraffin | 5.00 | 5.00 | 5.00 | 5.00 | 5.00 | 5.00 | 5.00 | 5.00 |
| | Monoethanolamine | 4.80 | 4.80 | 4.80 | 4.80 | 4.80 | 4.80 | 4.80 | 4.80 |
| | Para-aminophenol | 0.10 | 0.10 | 0.10 | 0.10 | 0.10 | 0.10 | 0.10 | 0.10 |
| | Paraphenylenediamine | 2.00 | 2.00 | 2.00 | 2.00 | 2.00 | 2.00 | 2.00 | 2.00 |
| | Meta-aminophenol | 0.20 | 0.20 | 0.20 | 0.20 | 0.20 | 0.20 | 0.20 | 0.20 |
| | Meta-phenylenediamine sulfate | 0.30 | 0.30 | 0.30 | 0.30 | 0.30 | 0.30 | 0.30 | 0.30 |
| | Resorcin | 0.50 | 0.50 | 0.50 | 0.50 | 0.50 | 0.50 | 0.50 | 0.50 |
| | Stearyltrimethylammonium chloride | 0.70 | 0.70 | 0.70 | 0.70 | 0.70 | 0.70 | 0.70 | 0.70 |
| | Stearic acid | 0.50 | 0.50 | 0.50 | 0.50 | 0.50 | 0.50 | 0.50 | 0.50 |
| | Poly N,N-dimethyl-3,4-dimethylene- | 0.42 | 0.42 | 0.42 | 0.42 | 0.42 | 0.42 | 0.42 | 0.42 |
| | pyrrolidinium chloride | | | | | | | | |
| | Pentasodium diethylenetriamine | 0.92 | 0.92 | 0.92 | 0.92 | 0.92 | 0.92 | 0.92 | 0.92 |
| | pentaacetate | | | | | | | | |
| | Sodium sulfite | 0.20 | 0.20 | 0.20 | 0.20 | 0.20 | 0.20 | 0.20 | 0.20 |
| | L-ascorbic acid | 0.50 | 0.50 | 0.50 | 0.50 | 0.50 | 0.50 | 0.50 | 0.50 |
| | Magnesium sulfate | 1.00 | 1.00 | 1.00 | 1.00 | 1.00 | 1.00 | 1.00 | 1.00 |
| | DL-sodium malate | 1.00 | 1.00 | 1.00 | 1.00 | 1.00 | 1.00 | 1.00 | 1.00 |
| | Purified water | 75.26 | 75.26 | 75.26 | 75.26 | 75.26 | 75.26 | 75.26 | 75.26 |
| | Total | 100.00 | 100.00 | 100.00 | 100.00 | 100.00 | 100.00 | 100.00 | 100.00 |
| Ingredient name (Second agent) | | | | | | | | | |
| (A) | Polyoxyethylene (10) cetyl ether | 1.00 | 1.00 | 1.00 | 1.00 | 1.00 | 1.00 | 1.00 | 1.00 |
| (A) | Polyoxyethylene (20) cetyl ether | 1.50 | 1.50 | 1.50 | 1.50 | 1.50 | 1.50 | 1.50 | 1.50 |
| (B) | Cetanol | 3.70 | 3.70 | 3.70 | 3.70 | 3.70 | 3.70 | 3.70 | 3.70 |
| (B) | Stearyl alcohol | 1.20 | 1.20 | 1.20 | 1.20 | 1.20 | 1.20 | 1.20 | 1.20 |
| (C) | Liquid paraffin | 0.10 | 1.00 | 8.00 | 10.00 | — | — | — | — |
| (C) | Liquid isoparaffin | — | — | — | — | 2.00 | — | — | — |
| (C) | Squalane | — | — | — | — | — | 2.00 | — | — |
| (C) | Vaseline | — | — | — | — | — | — | 2.00 | — |
| | Hydrogen peroxide | 5.81 | 5.81 | 5.81 | 5.81 | 5.81 | 5.81 | 5.81 | 5.81 |
| | Polyethylene glycol 400 | 1.50 | 1.50 | 1.50 | 1.50 | 1.50 | 1.50 | 1.50 | 1.50 |
| | Hydroxyethane diphosphonic acid | 0.08 | 0.08 | 0.08 | 0.08 | 0.08 | 0.08 | 0.08 | 0.08 |
| | Phosphoric acid | 0.02 | 0.02 | 0.02 | 0.02 | 0.02 | 0.02 | 0.02 | 0.02 |
| | Sodium monohydrogen phosphate | 0.10 | 0.10 | 0.10 | 0.10 | 0.10 | 0.10 | 0.10 | 0.10 |
| | Purified water | 84.99 | 84.09 | 77.09 | 75.09 | 83.09 | 83.09 | 83.09 | 85.09 |
| | Total | 100.00 | 100.00 | 100.00 | 100.00 | 100.00 | 100.00 | 100.00 | 100.00 |
| Evaluation | Simultaneous dispensability of the | A | A | A | B | A | A | B | A |
| items | first agent and the second agent | | | | | | | | |
| | Mixability of the first agent | B | A | A | B | B | B | B | C |
| | and the second agent | | | | | | | | |

From Examples 30 to 36 shown in Table 6, good results were obtained for the term storage.

Table 7 lists the results of the first agents and the second agents of the hair dye compositions prepared by the usual method in Examples 37 to 39.

The first agents and the second agents obtained according to Examples 37 to 39 exhibited good results with respect to the simultaneous dispensability and the mixability of the first agent and the second agent after long-term storage.

TABLE 7

| | | Exam- | Exam- | Exam- |
|---|---|---|---|---|
| | | ple 37 | ple 38 | ple 39 |
| Ingredient name (First agent) | | | | |
| (A) | Polyoxyethylene (20) stearyl | 2.70 | 2.70 | 2.70 |
| | ether | | | |
| (A) | Polyoxyethylene (5) behenyl | 1.30 | 1.30 | 1.30 |
| | ether | | | |
| (B) | Cetanol | 0.10 | 0.10 | 0.10 |
| (B) | Cetostearyl alcohol | 2.54 | 2.54 | 2.54 |
| (C) | Liquid paraffin | 5.00 | 5.00 | 5.00 |
| | Monoethanolamine | 4.80 | 4.80 | 4.80 |
| | Paraphenylenediamine | 2.16 | 2.00 | 2.00 |
| | Resorcin | 0.72 | 0.50 | 0.50 |
| | Para-aminophenol | 0.26 | 0.10 | 0.10 |
| | Meta-aminophenol | 0.20 | 0.20 | 0.20 |
| | Meta-phenylenediamine sulfate | — | 0.10 | 0.30 |
| | 2,4-Diaminophenoxyethanol | — | 0.20 | — |
| | hydrochloride | | | |
| | Stearyltrimethylammonium | 0.70 | 0.70 | 0.70 |
| | chloride | | | |
| | Stearic acid | 0.50 | 0.50 | 0.50 |
| | Poly N,N-dimethyl-3.4- | 0.42 | 0.42 | 0.42 |
| | dimethylene pyrrolidinium | | | |
| | chloride | | | |
| | Camellia oil | 0.01 | 0.01 | 0.01 |
| | Shea butter | 0.01 | 0.01 | 0.01 |
| | Ethanol | 0.24 | 0.24 | 0.24 |
| | 1,3-Butylene glycol | 0.10 | 0.10 | 0.10 |
| | Polyethylene glycol 200 | 0.25 | 0.25 | 0.25 |
| | Pentasodium diethylenetriamine | 0.92 | 0.92 | 0.92 |
| | pentaacetate | | | |
| | Sodium sulfite | 0.20 | 0.20 | 0.20 |
| | L-ascorbic acid | 0.50 | 0.50 | 0.50 |
| | DL-sodium malate | 1.00 | 1.00 | 1.00 |
| | Magnesium sulfate | 1.00 | 1.00 | 1.00 |
| | Citric acid | 0.01 | 0.01 | 0.01 |
| | Sodium citrate dihydrate | 0.01 | 0.01 | 0.01 |
| | Methyl parahydroxybenzoate | 0.01 | 0.01 | 0.01 |
| | Soy protein hydrolysate | 0.03 | 0.03 | 0.03 |
| | Royal jelly extract | 0.01 | 0.01 | 0.01 |
| | Eucheuma serra extract | 0.01 | 0.01 | 0.01 |
| | Grateloupia sparsa extract | 0.01 | 0.01 | 0.01 |
| | Saccharina angustata extract | 0.01 | 0.01 | 0.01 |
| | Ulva linza extract | 0.01 | 0.01 | 0.01 |
| | Undaria pinnatifida extract | 0.01 | 0.01 | 0.01 |
| | Licorice extract | 0.07 | 0.07 | 0.07 |
| | Collagen | 0.01 | 0.01 | 0.01 |
| | Purified water | 74.17 | 74.41 | 74.41 |
| | Total | 100.00 | 100.00 | 100.00 |
| | Viscosity of the first agent | 5,700 | 6,000 | 6,000 |
| | pH of the first agent | 10.5 | 10.5 | 10.5 |
| Ingredient name (Second agent) | | | | |
| (A) | Polyoxyethylene (10) cetyl ether | 0.95 | 0.95 | 0.95 |
| (A) | Polyoxyethylene (20) cetyl ether | 1.52 | 1.52 | 1.52 |
| (B) | Cetanol | 3.70 | 3.70 | 3.70 |
| (B) | Stearyl alcohol | 1.20 | 1.20 | 1.20 |
| (C) | Liquid paraffin | 2.00 | 2.00 | 2.00 |
| | Hydrogen peroxide | 5.81 | 5.81 | 5.81 |
| | Diethylene glycol monoethyl | 0.02 | 0.02 | 0.02 |
| | ether | | | |
| | Polyethylene glycol 400 | 1.50 | 1.50 | 1.50 |
| | Hydroxyethane diphosphonic | 0.08 | 0.08 | 0.08 |
| | acid | | | |
| | Phosphoric acid | 0.02 | 0.02 | 0.02 |
| | Sodium monohydrogen | 0.10 | 0.10 | 0.10 |
| | phosphate | | | |
| | Phenoxyethanol | 0.04 | 0.04 | 0.04 |
| | Purified water | 83.06 | 83.06 | 83.06 |
| | Total | 100.00 | 100.00 | 100.00 |
| | Viscosity of the second agent | 6,500 | 6,500 | 6,500 |
| | pH of the second agent | 3.0 | 3.0 | 3.0 |
| | Viscosity at the time of mixing | 5,100 | 5,200 | 5,000 |
| | pH at the time of mixing | 9.9 | 10.0 | 9.8 |

The present disclosure relates to an aerosol type cream hair dye composition, and according to which particularly the aerosol type cream hair dye composition, in which the first agent and the second agent have good simultaneous dispensability and good mixability after long-term storage, can be obtained.

What is claimed is:

1. An aerosol type cream hair dye composition comprising a first agent containing an alkaline agent and an oxidizing dye and a second agent containing an oxidizing agent, wherein both the first agent and the second agent contain (A) a nonionic surfactant, (B) a higher alcohol having 12 to 24 carbon atoms, and (C) a hydrocarbon in a liquid or paste form at 25° C.
